(19) United States
(12) Patent Application Publication
(10) Pub. No.: US 2008/0248116 A1
(43) Pub. Date: Oct. 9, 2008

(54) COMPOSITION FOR STIMULATING DE NOVO BONE INDUCTION

(75) Inventor: Ugo Ripamonti, Constantia Kloof (ZA)

Correspondence Address: DARBY & DARBY P.C., P.O. BOX 770, Church Street Station, New York, NY, 10008-0770, US

(73) Assignees: UNIVERSITY OF WITWATERSRAND, Parktown (ZA); MEDICAL RESEARCH COUNCIL OF SOUTH AFRICA, Cape Town (ZA)

(21) Appl. No.: 12/138,301

(22) Filed: Jun. 12, 2008

Related U.S. Application Data
(63) Continuation of application No. 10/947,394, filed on Sep. 22, 2004.
Application No. 10/947,394 is a continuation of application No. PCT/IB2003/001021, filed on Mar. 20, 2003.

(30) Foreign Application Priority Data
Mar. 20, 2002 (ZA) 2002/2307

(51) Int. Cl. A61K 9/10; A61K 38/18; A61P 19/00

(52) U.S. Cl. 424/484; 514/12

(57) ABSTRACT

This invention relates to a composition for stimulating de novo bone induction in a mammal, to the use of this composition in stimulating de novo bone induction in a mammal and to a method of treating a mammal to stimulate de novo bone induction. The use and method involve introducing the composition, preferably by local injection, into the mammal at a site where de novo bone induction is desired. The composition consists of a combination of a bone morphogenetic protein and a reconstituted basement membrane which, in a preferred embodiment of the invention is Matrigel®. The Matrigel® serves, at least partly, to retain the bone morphogenetic protein at the site of introduction for a period sufficient to trigger a bone differentiation cascade.

### FIELD OF THE INVENTION

This invention relates to a composition for initiating de novo bone induction, to a delivery system for initiating de novo bone induction and to a method of treating a mammal so as to initiate de novo bone induction.

### BACKGROUND TO THE INVENTION

Degenerative bone diseases and bone defects are relatively common and they are debilitating. Perhaps the most common example of one such disease is osteoporosis which tends to affect post-menopausal and hormonally deficient woman, and elderly men. Osteoporosis is a condition manifest in the reduction in the protein and mineral content of bone tissue which results in fragile and porous bones. As a result the bones tend to break easily and they also do not heal as rapidly as healthy bone. In severe conditions a sufferer a may need to have his or her joints replaced at regular intervals. Treatment by replacement hormone therapy can be effective, particularly in woman who are relatively young and have had an hysterectomy, but such treatment is chronic in that the patient must ingest hormonally preparations for the rest of her life. In addition, hormone replacement therapy may not always be effective. Consequently a composition that initiates de novo formation of healthy bone could be used to treat the majority of such persons and reduce their dependency on chronic hormonal preparations and/or surgical procedures.

A composition that initiates de novo formation of healthy bone could also be used in situations where bones are traumatized such as, for example, in a motor vehicle accident, while playing sport or during certain surgical procedures. Such surgical procedures can involve the removal of a portion of the iliac crest of the pelvic bone to graft into a bony defect where it acts as an autogenous bone graft to repair any one of several bone defects in a variety of bones. This technique is widely employed by orthopedic surgeons, surgeons involved in reconstructive surgery and maxillofacial surgeons. Another example involves cosmetic surgery where bones are displaced or deliberately fractured. A third example is provided by certain periodontal procedures which expose the roots and/or dentine of teeth such as in periodontal regenerative surgery and a fourth example is also provided by dental procedures where dental cavities are formed as a result of caries.

The use of bone morphogenetic and osteogenic proteins in inducing de novo formation of bone is well known in medical science. To induce bone formation effectively these proteins should be administered where bone formation is needed in combination or in conjunction with a carrier matrix so as to trigger a bone differentiation cascade. In 2000, direct adenoviral gene therapy was used to produce spinal fusion in rodents. The virus vector was injected directly into the fractured or osteoporotic vertebrae and this resulted in a de novo bone formation at the injection site. This method of stiffening bone formation does have a disadvantage the for its effectiveness in sites having healthy blood flow may be limited for it is conceivable that the vector can be washed out into the peripheral circulation before adequate transfection has occurred.

It is generally accepted that bone morphogenetic proteins must, to be effective, be delivered and remain in situ for a sufficient time to trigger the cascade of sequential events that lead to de novo bone induction.

Please note that the term “bone morphogenetic protein” as used in this specification is used, unless the context indicates otherwise, to refer to any one or more of a class of proteins that have been demonstrated to be effective in inducing de novo formation of bone and includes bone morphogenetic proteins, osteogenic proteins, and transformation growth factors—betas. All of which are know to persons skilled in this art as osteogenic members of the TGF-β superfamily.

### OBJECT OF THE INVENTION

Is an object of this invention to provide a composition for stimulating de novo bone induction and to provide a delivery system for initiating de novo bone induction.

### SUMMARY OF THE INVENTION

In accordance with this invention is provided a composition for stimulating de novo bone induction comprising a combination of a bone morphogenetic protein and a reconstituted basement membrane which is in a gel form at the normal body temperature of the animal to be treated, said composition being introducible into the body of an animal to be treated at a site where the de novo induction of bone is desired, the reconstituted basement membrane serving, at least partly, to retain the bone morphogenetic protein at said site for a period sufficient to trigger a bone differentiation cascade.

There is further provided for the composition to include an osteogenic protein, alternatively a transformation growth factor-beta and for the bone morphogenetic protein to be selected from the group comprising a recombinant human bone morphogenetic protein, an osteogenic protein and transformation growth factor beta.

There is also provided for the reconstituted basement membrane to be a growth factor reduced matrix, for the matrix to predominantly contain laminin, type IV collagen and heparan sulphate proteoglycan, and for the reconstituted basement membrane to be a MATRIGEL® matrix. Alternatively there is provided for the growth factor reduced matrix to be manufactured by combining extra cellular matrix components produced by recombinant DNA technology.

There is further provided for the composition to be used to stimulate de novo formation of bone in cases where the animal to be treated has suffered accidental trauma to its bones, alternatively where the animal to be treated has suffered surgical trauma to its bones, further alternatively where the animal to be treated is suffering from a degenerative bone disorder such as, for example, osteoporosis and still further preferably a bone defect, and for the animal to be treated to be a mammal, preferably a primate and further preferably a human.

The invention also provides for a method of treating a mammal suffering from a degenerative bone disorder or trauma to its bones, said method comprising introducing, into the body of the mammal to be treated at a site where the de novo induction of bone is desired, a composition for stimulating de novo bone induction comprising a combination of a bone morphogenetic protein and a reconstituted basement membrane which is in a gel form at the normal body temperature of the mammal, the reconstituted basement membrane serving, at least partly, to retain the bone morphogenetic protein at the site of introduction for a period sufficient to trigger a bone differentiation cascade.

There is further provided for the composition to include an osteogenic protein, alternatively a transformation growth factor beta and for the bone morphogenetic protein to be a recombinant human bone morphogenetic protein, osteogenic protein or transformation growth factor-beta.

There is also provided for the reconstituted basement membrane to be a growth factor reduced matrix, for the matrix to predominantly contain laminin, type IV collagen and heparan sulphate proteoglycan, and for the reconstituted basement membrane to be a MATRIGEL® matrix.

There is further provided for the degenerative bone disorder or trauma from which the mammal is suffering to be skeletal defects, alternatively periodontal defects and, further alternatively, systemic bone loss such as, for example, osteoporosis and for the method to provide for local injection of osteogenic members of the transforming growth factor-beta superfamily to locally treat skeletal defects, non-union fractures and bones affected by systemic bone loss.

The invention also provides for the mammal to be treated to be a primate and, preferably, a human.

### BRIEF DESCRIPTION OF THE EXAMPLES

Embodiments of the invention will be described below by way of example only and with reference to the accompanying examples in which:

Example 1 is a rodent bioassay demonstrating the osteogenic activity and delivery of bone morphogenetic proteins and osteogenic proteins by basement membrane MATRIGEL®; and,

Example 2 demonstrates a novel method for de novo induction of bone in ovariectomized primates Papio ursinus affected by systemic bone loss.

### DETAILED DESCRIPTION OF THE EXAMPLES

Example 1A Rodent Bioassay Demonstrating the Osteogenic Activity and Delivery of Bone Morphogenetic Proteins and Osteogenic Proteins or any Other Human Recombinant Bone Morphogenetic Protein and Transforming Growth Factor-Beta by Basement Membrane MATRIGEL®.

To study the osteogenic activity and delivery of bone morphogenetic proteins by basement membrane Matrigel a heterotopic rodent bioassay was selected. Aliquots of the naturally-derived soluble signals, bone morphogenetic proteins (BMP) and osteogenic proteins (OP), were extracted and purified after gel filtration chromatography. Alternatively recombinant human osteogenic protein (hOP-1) could be used. Doses of 2 and 4 μg (hOP-1) were dissolved in 5 mM HCl and added to basement membrane Matrigel® at a concentration of 250 μl Matrigel per dose of soluble signals. Reconstituted stock solutions of basement membrane Matrigel were kept fluid on ice, vortexed and aliquots of 250 μl were recovered using 500 μl pipettes. These aliquots were injected into the subcutaneous space at bilateral sites over the pectoralis fascia of anaesthetized 28-35 days-old Long Evans rats.

A total of 14 Long Evans rats were used and implanted with 16 basement membrane Matrigel with 20 μl osteogenic fractions of naturally-derived BMPs/OPs after gel filtration chromatography and 8 basement membrane Matrigel to which 2 μg and 4 μg hOP-1 in 5 mM HCl were added. Basement membrane Matrigel, 250 μl but without soluble signals was also implanted as control. Skin incisions were closed in one layer using atraumatic 2-0 resorbable sutures. Implants were harvested on day 12 and generated ossicles were fixed in ethanol, infiltrated and embedded in Historesin. Sections, cut at 2 μm, were stained with toluidine blue.

At harvest, basement membrane Matrigel reconstituted with doses of naturally-derived BMPs/OPs and hOP-1 resulted in firm and enlarged highly vascularized red ossicles. In all instances, the histological analysis showed a reprodible pattern of extensive endochondral bone differentiation, vascular invasion and remodelling of the newly formed ossicles. Doses of 4 μg hOP-1 induced substantial haemopoietic bone marrow with marked chondrolysis.

Example 2A Novel Method for De Novo Induction of Bone in Ovariectomized Primates Papio ursinus Affected by Systemic Bone Loss.

In a cohort of long-term ovariectomized (OVX) primates, Papio ursinus, morphological and structural iliac crest histomorphometric data 36 months post-OVX showed that bone trabecular morphology was permanently lost after OVX. Bone mineral density (BMD) measurements of the lumbar spine showed L3 and is L4 more intensely affected by oestrogen depletion than other skeletal areas examined. After the identification of the basement membrane Matrigel as carrier for the osteogenic activity of BMPs/OPs, it was envisaged that the direct injection of naturally-derived BMPs/OPs would initiate bone formation in the affected vertebral bodies with recovery of the original trabecular morphology.

The above study was conducted according to the Guidelines for the Care and Use of Experimental Animals prepared by the University of the Witwatersrand and in compliance with the National Code for Animal Use in Research, Education and Diagnosis in South Africa. Before any procedures were initiated, research protocols were approved by the Animal Ethics Screening Committee of the University (Clearance Certificate 2000/3515). Two adult females weighing 26.2 and 20.7 kg respectively, were selected from the Units's cohort of 24 OVX primates Papio ursinus. Animals were immobilized and anaestherised and, after surgical preparation of the injection site, a pediatric bone marrow biopsy needle was inserted through the posterior wall of the third and forth vertebral body with the baboon in lateral position, 2-2.5 cm off the midline, 5 cm below the superior edge of the spinous process at an angle between 35 and 40° off the midline considering the sagittal plane of the vertebra. Growth factors-reduced (Gfr) Matrigel Matrix was used to absorb and deliver naturally-derived BMPs/OPs extracted and purified from bovine bone matrix. Total amount injected into each vertebra was 1 ml Gfr-Matrigel and 0.5 ml of 5 mM hydrochloric acid containing 500 μg of naturally-derived BMPs/OPs. Animals fully recovered from the experiment and no signs of neurological deficits were noted immediately after experimentation nor during later observation.

On day 0, 30 and 90 bone mineral density (BMD) of selected skeletal sites was measured by dual photon x-ray absorptionmetry (DXA). Lateral (L2, 3 and 4) vertebral BMD was measured by the Hologic QDR 4500/A osteodensitometer. Before lateral BMD measurements, we investigated the anatomy of vertebral body sections from a group of euthanased adult female baboon which were part of different studies. Lumbar vertebral dimension was measured on lateral x-ray of experimental animals and on post-mortem specimens of adult female baboons lumbar vertebrae obtained from different experiments. Areas of L1, 2, 3 and 4 were measured and data were used to analyse lateral BMD images. Only the central areas of the vertebra was analysed on the later image, allowing a reproducible analysis of the cancellous bone. The transverse axis was similar in all four vertebrae while the longitudinal axis was longer in L3 and L4 (Table 1) and the region of interest area (ROI) was also greater in L3 and L4 (Table 2). L2 was not injected. Baboon 1, L2 showed a decreased BMD (−6.1%) 30 days after the local injection followed by a gain (9.3%) on day 90. Baboon 2, L2 showed a steady BMD on day 30 and a positive gain (10.3%) on day 90. L3 of animal 1 showed a rapid bone loss (−23%) after injection which was followed by a rapid bone gain (38%) as measured 90 days after injection. Compared to baseline, L3 BMD 90 days after injection was 20% higher. Animal 2 showed a rapid bone loss in L3 (−9.4%) and only a minimal trend of recovery (0.9%) during 90 days observation. L4 of animal 1 BMD was markedly increased 30 days post-injection (41%) but this was followed by 60 days of rapid bone loss (−49%) which resulted in a net 13.7% negative difference compared to baseline. Animal 2 L$ BMD showed a steady increase during observations, 8.3% and 11.7% at 30 and 90 days respectively.

Although only four vertebrae in two animals were injected, results showed that bone remodelling has changed in the treated lumbar vertebrae and the trend of bone loss on day 30 after injection in L3 was followed by a period of rapid recovery detected 2 months later. This may be the result of rapid remodelling followed by bone formation by induction. At this stage it may be premature to formulate mechanistic hypotheses on bone behaviour for these non linear trend, nevertheless these changes were important and promptly detected by lateral BMD measurements. Although L2 was not injected, an increased BMD in both animals were detected on day 90. It is likely that events in nearby vertebrae propagated signals to initiate bone formation to untreated bones through the is circulatory system. Although we were not able to follow the distribution of reconstituted basement membrane Matrigel within the injected vertebral bodies nor the time of exposure of BMPs/OPs with responding cells it is, nevertheless, believed that this therapeutic approach, particularly the direct injection of basement membrane Matrigel reconstituted with BMPs/OPs acting as a novel soluble delivery system is an innovative approach for the treatment of established osteoporosis in clinical contexts as well as a novel delivery system for BMPs/OPs and transforming growth factor betas to initiate bone formation by induction in a variety of skeletal defects.

It will be appreciated that, while example relates to a method of de novo bone induction in primates, the techniques described can be extended the induction of de novo bone formation in humans.

1. A composition for stimulating de novo bone induction comprising a combination of a bone morphogenetic protein or a transformation growth factor beta protein and a reconstituted basement membrane comprising a growth factor reduced matrix, which predominantly contains laminin, type IV collagen, and heparan sulphate proteoglycan, and which is in a gel form at the normal body temperature of an animal to be treated, said composition being introducible into the body of an animal to be treated at a site of bone damage or degeneration where the de novo induction of bone is desired, the reconstituted basement membrane serving, at least partly, to retain the bone morphogenetic protein at said site for a period sufficient to trigger a bone differentiation cascade.

2. (canceled)

3. (canceled)

4. A composition for stimulating de novo bone induction as claimed in claim 1 in which the bone morphogenetic protein or transformation growth factor beta protein is human recombinant protein.

5. (canceled)

6. (canceled)

7. A composition for stimulating de novo bone induction as claimed in claim 1 in which the reconstituted basement membrane is a MATRIGEL matrix.

8. A composition for stimulating de novo bone induction as claimed in claim 1 in which the reconstituted basement membrane is manufactured by combining extra cellular matrix components produced by recombinant DNA technology.

9. A composition for stimulating de novo bone induction as claimed in claim 1 in which the animal to be treated is a mammal.

10. A composition for stimulating de novo bone induction as claimed in claim 9 in which the mammal is a primate.

11. A composition for stimulating de novo bone induction as claimed in claim 10 in which the primate is a human.

12. Use of a composition for stimulating de novo bone induction as claimed in claim 1 to stimulate de novo formation of bone in an animal that has suffered accidental trauma to its bones comprising introducing said composition into the body of the animal at or close to the site of trauma.

13. Use of a composition for stimulating de novo bone induction as claimed in claim 1 to stimulate de novo formation of bone in an animal that has suffered surgical trauma to its bones comprising introducing said composition into the body of the animal at or close to the site of trauma.

14. Use of a composition for stimulating de novo bone induction as claimed in claim 1 to stimulate de novo formation of bone in an animal that is suffering from a degenerative bone disorder such as, for example, osteoporosis comprising introducing said composition into the body of the animal at or close to the site of bone suffering from said degenerative bone disorder.

15. A method of treating a mammal suffering from a degenerative bone disorder or trauma to its bones, said method comprising introducing, into the body of the mammal to be treated at a site where the de novo induction of bone is desired, a composition for stimulating de novo bone induction comprising a combination of a bone morphogenetic protein and a reconstituted basement membrane which is in a gel form at the normal body temperature of the mammal, the reconstituted basement membrane serving, at least partly, to retain the bone morphogenetic protein at the site of introduction for a period sufficient to trigger a bone differentiation cascade.

16. A method of treating a mammal suffering from a degenerative bone disorder or trauma to its bones as claimed in claim 15 in which the composition for stimulating de novo bone induction includes an osteogenic protein.

17. A method of treating a mammal suffering from a degenerative bone disorder or trauma to its bones as claimed in claim 15 in which the composition for stimulating de novo bone induction includes a transformation growth factor beta.

18. A method of treating a mammal suffering from a degenerative bone disorder or trauma to its bones as claimed in claim 15 in which the bone morphogenetic protein component of said composition is selected from the group comprising a recombinant human bone morphogenetic protein, an osteogenic protein and transformation growth factor beta.

19. A method of treating a mammal suffering from a degenerative bone disorder or trauma to its bones as claimed in claim 15 in which the reconstituted basement membrane component of the composition is a growth factor reduced matrix.

20. A method of treating a mammal suffering from a degenerative bone disorder or trauma to its bones as claimed in claim 19 in which the matrix predominantly contains laminin, type IV collagen and heparan sulphate proteoglycan.

21. A method of treating a mammal suffering from a degenerative bone disorder or trauma to its bones as claimed in claim 15 in which the reconstituted basement membrane component of the composition is a MATRIGEL matrix.

22. A method of treating a mammal suffering from a degenerative bone disorder or trauma to its bones as claimed in claim 15 in which the degenerative bone disorder or trauma from which the mammal is suffering are skeletal defects.

23. A method of treating a mammal suffering from a degenerative bone disorder or trauma to its bones as claimed in claim 15 in which the degenerative bone disorder or trauma from which the mammal is suffering are periodontal defects.

24. A method of treating a mammal suffering from a degenerative bone disorder or trauma to its bones as claimed in claim 15 in which the degenerative bone disorder or trauma from which the mammal is suffering are caused by systemic bone loss such as, for example, osteoporosis.

25. A method of treating a mammal suffering from a degenerative bone disorder or trauma to its bones as claimed in claim 15 in which the method to osteogenic members of the transforming growth factor-beta superfamily are injected locally to locally treat skeletal defects, non-union fractures and bones affected by systemic bone LOSS.

26. A method of treating a mammal suffering from a degenerative bone disorder or trauma to its bones as claimed in claim 15 in which the reconstituted basement membrane is a product of a combination of extra cellular matrix components produced by recombinant DNA technology.
